(19) United States
(12) Patent Application Publication
(10) Pub. No.: US 2021/0228313 A1
(43) Pub. Date: Jul. 29, 2021

(54) METHOD AND APPARATUS TO ACCOUNT FOR TRANSPONDER TAGGED OBJECTS DURING CLINICAL PROCEDURES, EMPLOYING OPERATIVE SITE SURGICAL DRAPE WITH INTEGRATED ANTENNA

(71) Applicant: Covidien LP, Mansfield, MA (US)

(72) Inventors: Allan G. Aquino, Longmont, CO (US); David Keppel, Longmont, CO (US); Andy Buersmeyer, Ft. Collins, CO (US); Richard L. Croft, Mead, CO (US)

(21) Appl. No.: 17/159,734

(22) Filed: Jan. 27, 2021

Related U.S. Application Data
(60) Provisional application No. 62/966,667, filed on Jan. 28, 2020.

(51) Int. Cl. A61B 90/98; A61B 46/20; G16H 40/40

(52) U.S. Cl. CPC A61B 90/98; G16H 40/40; A61B 46/20

(57) ABSTRACT

A surgical drape, configured for in situ deployment on a patient, includes a panel having an outer surface and an inner surface; a fenestration centrally located in and extending through the panel; and an antenna connected to the panel, wherein the antenna surrounds the fenestration.

### CROSS-REFERENCE TO RELATED APPLICATIONS

This application claims the benefit of and priority to U.S. Provisional Patent Application Ser. No. 62/966,667 filed Jan. 28, 2020, the entire disclosure of which is incorporated by reference herein.

### TECHNICAL FIELD

This disclosure relates to operative site surgical drapes, and more specifically to methods and apparatus to account for transponder tagged objects during clinical procedures, employing operative site surgical drapes with integrated antenna.

### BACKGROUND

It is often useful or important to determine whether objects associated with a surgery are present in a patient's body before completion of the surgery. Such objects may take a variety of forms. For example, the objects may take the form of instruments, for instance scalpels, scissors, forceps, hemostats, and/or clamps. Also for example, the objects may take the form of related accessories and/or disposable objects, for instance surgical sponges, gauzes, and/or pads. Failure to locate an object before closing the patient may require additional surgery, and in some instances may have serious adverse medical consequences.

Some hospitals have instituted procedures which include checklists or requiring multiple counts to be performed to track the use and return of objects during surgery. Such a manual approach is inefficient, requiring the time of highly trained personnel, and is prone to error.

Another approach employs transponders and a wireless interrogation and detection system. Such an approach employs wireless transponders (e.g., RF tags) which are attached to various objects used during surgery. The interrogation and detection system includes a transmitter that emits pulsed wideband wireless signals (e.g., radio or microwave frequency) and a detector for detecting wireless signals returned by the transponders in response to the emitted pulsed wideband signals. Such an automated system may advantageously increase accuracy while reducing the amount of time required of highly trained and highly compensated personnel. Examples of such an approach are discussed in U.S. Pat. No. 6,026,818, issued Feb. 22, 2000, and U.S. Patent Publication No. US 2004/0250819, published Dec. 16, 2004 (abandoned).

Commercial implementation of such an automated system requires that the overall system be cost competitive and highly accurate. In particular, false negatives must be avoided to ensure that objects are not mistakenly left in the patient. Some facilities may wish to install a single interrogation and detection system in each surgery theater, while other facilities may move an interrogation and detection system between multiple surgical theaters.

During a surgery, an operative site surgical drape is employed to maintain sterile conditions, maintain patient privacy, absorb fluid, and provide a clear and clean work area for the clinician. Some operative site surgical drapes are provided with an opening, window or fenestration formed therein that provides the clinician with access to a desired site on the patient's body while preserving the function of the surgical drape for other areas of the patient's body.

Accordingly, it is desired to have an operative site surgical drape including an antenna incorporated therein to enable detection of transponders on surgical items used on the patient before, during and after the surgery.

### SUMMARY

This disclosure relates to operative site surgical drapes for use with surgical objects and devices used in body cavities during surgery, which surgical objects have RF tags connected thereto or incorporated therein.

According to an aspect of the disclosure, a surgical drape is provided and is configured for in situ deployment on a patient. The surgical drape includes a panel having an outer surface and an inner surface; a fenestration centrally located in and extending through the panel; and an antenna connected to the panel, wherein the antenna surrounds the fenestration.

The antenna may be a coil of conductive material. The antenna may be planar. The antenna may be constructed of aluminum.

The antenna may be placed on the outer surface of the panel. The antenna may be a first antenna, and the surgical drape may include a second antenna connected to the panel and surrounding the fenestration. The second antenna may be placed on the inner surface of the panel.

The second antenna may be electrically connected to the first antenna.

The coils of the first antenna may be laterally offset from coils of the second antenna.

The antenna may be imbedded in the outer surface of the panel. The antenna may be a first antenna, and the surgical drape may include a second antenna connected to the panel and surrounding the fenestration. The second antenna may be imbedded in the inner surface of the panel.

The second antenna may be electrically connected to the first antenna.

The coils of the first antenna may be laterally offset from coils of the second antenna.

According to another aspect of the disclosure, an interrogation and detection system for use on a patient in a surgical environment is provided and includes a controller; a medical device having a transponder configured for storing information thereon; and a surgical drape configured for in situ deployment on the patient while in the surgical environment. The surgical drape includes a panel having an outer surface and an inner surface; a fenestration centrally located in and extending through the panel; and an antenna connected to the panel and connectable to the controller, the antenna surrounding the fenestration, wherein the antenna is configured to interrogate an area of the patient that is in proximity to the fenestration in response to control signals received from the controller. The antenna is configured to receive response signals from the transponder of the medical device and transmit the response signals to the controller for processing.

The antenna may be a coil of conductive material. The antenna may be planar. The antenna may be constructed of aluminum.

The antenna may be placed on the outer surface of the panel. The antenna may be a first antenna, and the surgical drape may include a second antenna connected to the panel and surrounding the fenestration. The second antenna may be placed on the inner surface of the panel. The controller may control each of the first and second antennas.

The second antenna may be electrically connected to the first antenna.

The coils of the first antenna may be laterally offset from coils of the second antenna.

The antenna may be imbedded in the outer surface of the panel. The antenna may be a first antenna, and the surgical drape may include a second antenna connected to the panel and surrounding the fenestration. The second antenna may be imbedded in the inner surface of the panel. The controller may control each of the first and second antennas.

The second antenna may be electrically connected to the first antenna.

The coils of the first antenna may be laterally offset from coils of the second antenna.

### BRIEF DESCRIPTION OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

Various embodiments of the presently disclosed operative site surgical drape are described herein below with reference to the drawings, wherein:

FIG. 1 is an illustration of an operating room environment depicting an operative site surgical drape of this disclosure draped over a patient while on an operating table;

FIGS. 2 and 3 are schematic illustrations showing prior art surgical objects have RF tags connected thereto or incorporated therein;

FIG. 4 is a schematic plan view of an operative site surgical drape according to the disclosure;

FIG. 5 is a schematic plan view of an antenna of the operative site surgical drape, from the indicated area of detail of FIG. 4;

FIG. 6 is a cross-sectional view of the operative site surgical drape of FIG. 4, as taken through 6-6 of FIG. 5; and

FIG. 7 is a cross-sectional view of the operative site surgical drape of FIG. 4, as taken through 6-6 of FIG. 5, illustrating another embodiment thereof.

### DETAILED DESCRIPTION OF EMBODIMENTS

In the following description, certain specific details are set forth in order to provide a thorough understanding of disclosed embodiments. However, one skilled in the relevant art will recognize that embodiments may be practiced without one or more of these specific details, or with other methods, components, materials, etc. In other instances, well-known structures associated with transmitters, receivers, or transceivers have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

As used herein, the terms parallel and perpendicular are understood to include relative configurations that are substantially parallel and substantially perpendicular up to about + or −10 degrees from true parallel and true perpendicular.

The headings and Abstract of the Disclosure provided herein are for convenience only and do not interpret the scope or meaning of the embodiments.

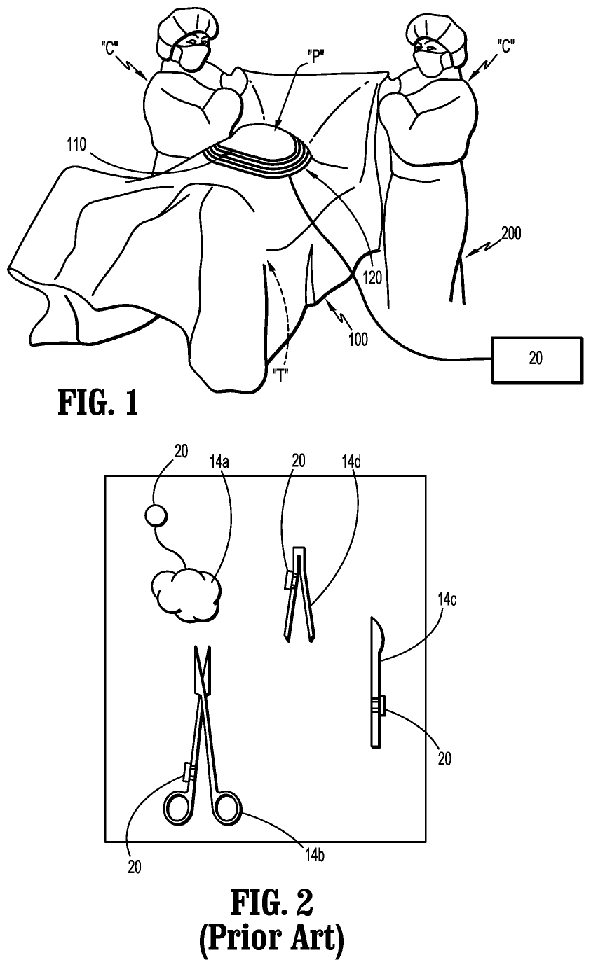

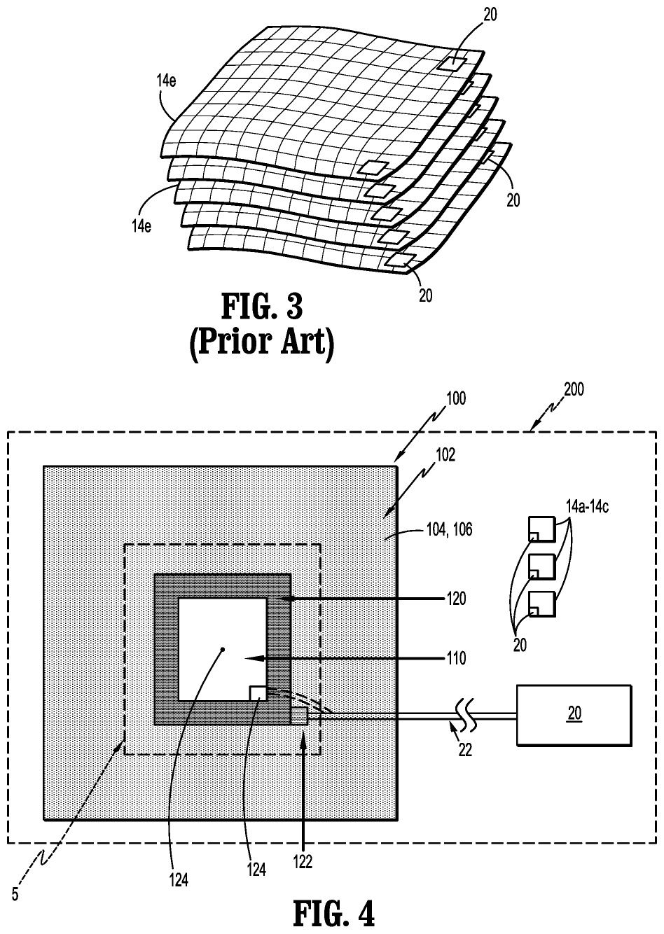

FIG. 1 depicts a surgical environment or operating room environment in which medical providers and clinicians “C” are operating on a patient “P” while the patient “P” is laying on a surgical table “T”, and is draped with an operative site surgical drape 100 of the disclosure.

The operative site surgical drape 100 can be used in a wide range of treatment situations such as surgery, medical device implantation procedures, and medical device servicing procedures. With reference to FIGS. 2A and 2B, examples of medical devices that may be used during a surgical procedure include, and are not limited to, cutting devices (e.g., a scalpel 14c, lancet, knife, scissors), grasping devices (e.g., tweezers 14d, forceps), clamping devices (e.g., hemostat 14b, clamps), access devices (e.g., dilators, specula), injection/irrigation devices (e.g., needles, tips), drilling devices (e.g., a drill bit), or measurement devices (e.g., rulers, calipers). In addition, the medical devices may also include sponges 14a, and/or absorbent surgical sponges, gauze and/or padding 14e. Each medical device 14a-14e includes an apparatus and/or transponder 20 attached, affixed, or otherwise coupled thereto.

The apparatus and/or transponder 20 may be constructed in various manners. For example, the apparatus and/or transponder 20 may include a ferrite rod with a conductive coil wrapped about an exterior surface thereof to form an inductor, and a capacitor coupled to the conductive coil to form a series circuit. The conductive coil may, for example, take the form of a spiral wound conductive wire with an electrically insulative sheath or sleeve. In other implementations, the apparatus and/or transponder 20 includes an RFID chip that stores identification information that uniquely identifies the apparatus and/or transponder 20. Additional details about types of apparatus and/or transponders 20 may be found in U.S. Patent Publication No. 2007-0285249; U.S. Pat. No. 8,710,957; and U.S. Pat. No. 9,717,565, each of which are herein incorporated by reference.

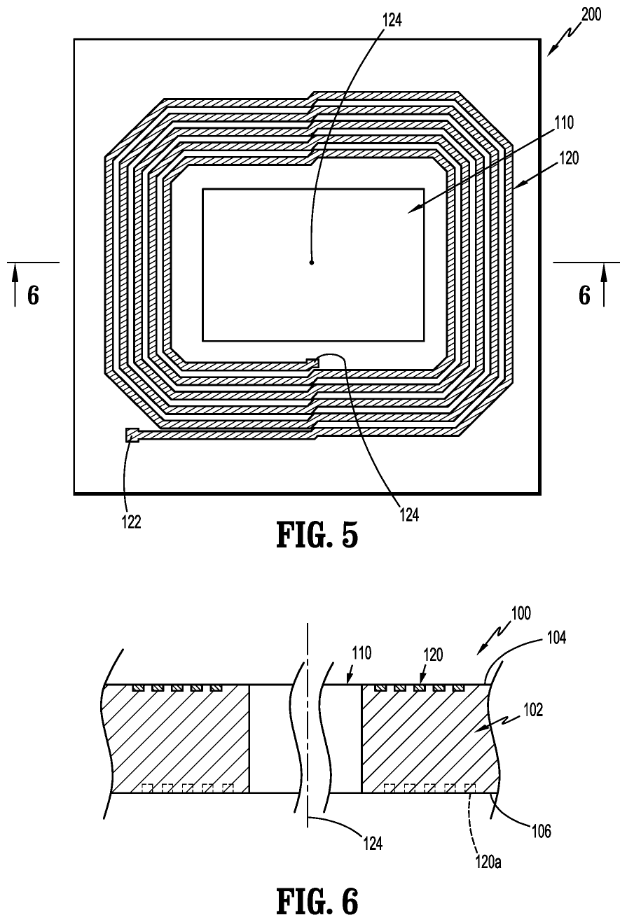

With reference to FIGS. 1 and 4-7, a surgical drape 100, with in situ features, is shown and refers to use of the surgical drape 100 directly on a patient “P” while in the surgical environment. The surgical drape 100 provides a sterile and clear working surface for the clinician “C” performing the procedure. The surgical drape 100 also assists in maintaining a patient's modesty, comfort, and privacy, as well as to isolate the surgical site from the other areas of the patient's body and nonsterile areas of the operating room and/or table “T”.

The surgical drape 100 includes a panel 102 having an outer surface 104, an inner surface 106, and a centrally located fenestration or window 110 formed in and through the panel 102. The panel 102 can take a wide variety of shapes such as a tetragon, including and not limited to a rectangle, square, polygon, trapezoid, or other geometry, for example, triangle, pentagon, hexagon, etc. The surgical drape 100 is sized for the intended surgical procedure and for the intended patient “P”, such as, for example, a 76.2 cm (30.0 in) tetragon square.

The panel 102 of the surgical drape 100 is made from material(s) suitable for surgery, including and not limited to cloth or paper, and which may be reusable or disposable. The inner surface 106 of the panel 102 may be coated with a thin nonabsorbent material such as a plastic polycoat to prevent or reduce a transfer of fluids across the panel 102. The panel 102 may be constructed from an electrically insulative material. Alternatively, the surgical drape 100 may include an electrically insulative substrate interposed between the panel 102 and the antenna 120.

The fenestration 110 is an opening in the panel 102 which is sized for the intended procedure. The fenestration 110 can be sized for a medical device such as, for example, a 8.9 cm (3.5 in) circular opening or rectangular opening. In an embodiment, the fenestration 110 may be about 24 inches. It is contemplated that the fenestration 110 may have a size and shape that is appropriate for a given surgical procedure/incision.

Although the fenestration 110 is generally centrally located in the panel 102, the fenestration 110 can be placed off center in the panel 102. The surgical drape 100 may also include a fenestration cover (not shown) that covers the fenestration 110 to aid in maintaining sterility and patient modesty until the clinician is ready to perform the desired procedure.

The surgical drape 100 may include an adhesive placed on the inner surface 106 of the panel 102, near the fenestration 110, which is of a type compatible with surgical use and of an amount sufficient to attach the surgical drape 100 to the patient “P” in a manner desired by the clinician “C”. For example, the adhesive can be at least one adhesive strip or more strips.

With continued reference to FIGS. 1 and 4-6, the surgical drape 100 includes an antenna 120 supported on the outer surface 104, the inner surface 106 of the panel 102 or disposed within the panel 102, between the outer surface 104 and the inner surface 106.

The antenna 120 may, for example, take the form of an annulus or air-coil formed of coils of conductive material having a planar profile. The conductive material may, for example, take the form of wire or may take the form of a conductive trace printed or otherwise deposited on the outer surface 104 or the inner surface 106 of the panel 102. While being formed of a conductive material, it is contemplated that the antenna 120 be formed of a radiolucent conductive material. For example, the antenna 120 may be formed as a thin (e.g., thickness, width) strip line aluminum antenna. The antenna 120 may be fabricated from any conductive material, such as, for example, and not limited to silver, copper, gold, aluminum, tungsten, zinc, nickel, iron, and stainless steel. The antenna 120 acts as an inductor.

The antenna 120 surrounds or circumscribes the fenestration 110 of surgical drape 120. The antenna 120 may include ten turns evenly spaced between an inner diameter of about 11 inches and an outer diameter of about 14 inches. In an embodiment, the antenna 120 may have a diameter of about 24 inches. It is contemplated that the antenna 120 may have a diameter that is appropriate for a given surgical procedure/incision. It is contemplated that the antenna 120 may include fewer or more than ten turns. It is further contemplated that the antenna 120 may have a rectangular configuration having an internal dimension of about 11 inches to about 14 inches. It is contemplated that the antenna 120 may have an internal dimension that is radially larger than a maximum cross-sectional dimension of the fenestration 110.

With specific reference to FIG. 6, antenna 120 may be embedded into the outer surface 104 of the panel 102, so as to be flush with the outer surface 104. It is contemplated that the surgical drape 100 may also include a second antenna 120a (shown in phantom) embedded into the inner surface 106 of the panel 102, and in registration with antenna 120. In such an arrangement, the coils of the second antenna 120a may be disposed between or interleaved with the coils of antenna 120.

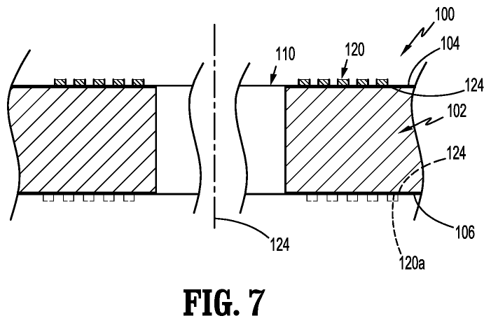

With specific reference to FIG. 7, antenna 120 may be a separate stand-alone component which is added-on or placed on top of the outer surface 104 of the panel 102, and adhered to the panel 102 via a layer of adhesive 124 or the like. It is contemplated that the surgical drape 100 may also include a second separate stand-alone antenna 120a (shown in phantom) which is placed on top of the inner surface 106 of the panel 102, and adhered to the panel 102 via a layer of adhesive 124 or the like. The second antenna 120a is in registration with antenna 120. In such an arrangement, the coils of the second antenna 120a may be disposed between or interleaved with the coils of antenna 120.

The antenna 120 may be electrically coupled to the second antenna 120a, for example, by a plug of electrically conductive material (not shown) which extends through a via or forms a via. In practice, the via connecting the antenna 120 to the second antenna 120a may include a vertical connector of the same material as the antennas 120, 120a. Any suitable conductor may be used to connect the antennas 120, 120a.

In configurations including antennas 120, 120a, as mentioned above, in addition to the antennas 120, 120a being offset from each other vertically along a central axis 124 thereof (see FIGS. 4-7), the individual windings of the antennas 120, 120a are also offset from each other laterally along two perpendicular axes in a horizontal plane (e.g., the plane of the drawing sheet of FIGS. 4-7) that is perpendicular to the central axis 124. Specifically, the antenna 120 is positioned relatively above the second antenna 120a. As noted above, the coil of the second antenna 120a is offset from the coil of the antenna 120 vertically along the central axis 124 as well as laterally in a horizontal plane which is perpendicular to the central axis 124. The individual windings of the coil of antenna 120 are spaced apart leaving gaps between each winding. The individual windings of the coil of the second antenna 120a are spaced directly below the gaps between the windings of the coil of the antenna 120. The two sets of windings are thus spread in such a way to provide a more even distribution of radiolucence. This distribution of the windings may smooth the contrast that could appear in a radiological image (e.g., X-ray image). Many other configurations of the coils and the windings of the antennas 120, 120a will be apparent to those of skill in the art and fall within the scope of this disclosure. The configuration of windings illustrated in FIGS. 6 and 7 are given only by way of example and do not limit the scope of the disclosure. The surgical drape 100 and antennas 120, 120a are not drawn to scale. Relative heights, widths, and separations of the coils of the antennas 120, 120a may be different in practice than what is shown in FIGS. 6 and 7.

It is contemplated that the surgical drape 100 may be provided with a cover (not shown) or the like which can overlay the antenna 120 and/or the antenna 120a. The cover may provide protection to the antenna(s) 120, 120a while the surgical drape 100 is being stored, transported, or used.

With reference back to FIG. 4, the antenna 120 includes a pair of terminals 122, 124 that provide electrical coupling to a controller 20, for example, via a coaxial cable 22. For a detailed discussion and illustration of an exemplary controller 20, for use with the surgical drape 100 and for sure in an interrogation and detection system, reference may be made to U.S. Pat. No. 9,792,408, the entire content of which is incorporated herein by reference.

The surgical drape 100, in combination with the medical devices 14a-14e (including RFID tags 20) and the controller 18, may form part of an interrogation and detection system 200 (see FIGS. 1 and 4). The interrogation and detection system is operable to ascertain the presence or absence of medical devices 14a-14e tagged with RFID tags or transponders 20, which may be in or on a patient “P”. Generally, the antenna 120 is connectable to the controller 20, and is configured to interrogate an area of the patient “P” (via transmission of wideband interrogation signals) that is in proximity to the fenestration 110 in response to control signals received from the controller 20. The antenna 120 is configured to receive signals from the transponder 20 of the medical device 14-14e and transmit those signals to the controller 20 for processing. For a detailed discussion and illustration of an exemplary interrogation and detection system, reference may be made to U.S. Pat. No. 9,792,408, the entire content of which was previously incorporated herein by reference.

The surgical drape 100 and/or interrogation and detection system 200 of the disclosure, allows for a more hands-free operation, as compared to predecessor systems and technologies. The surgical drape 100 and/or interrogation and detection system 200 of the disclosure eliminates the need for a hand-held scanner, eliminates operator scanning variability or technique inherent with hand held scanner use, provides for closer proximity to a surgical site allowing for reduced power level requirements of scanning or tag/transponder excitation; provides for closer proximity to surgical site allowing for increased sensitivity of transponder detection, provides for closer proximity to surgical site reducing environmental RF interference, and enables a greater number of surgical items to be tagged with a tag/transponder.

While an embodiment of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A surgical drape configured for in situ deployment on a patient, the surgical drape comprising:
a panel having an outer surface and an inner surface;
a fenestration centrally located in and extending through the panel; and
an antenna connected to the panel, wherein the antenna surrounds the fenestration.

2. The surgical drape according to claim 1, wherein the antenna is a coil of conductive material.

3. The surgical drape according to claim 2, wherein the antenna is planar.

4. The surgical drape according to claim 3, wherein the antenna is placed on the outer surface of the panel.

5. The surgical drape according to claim 4, wherein the antenna is a first antenna, and the surgical drape includes a second antenna connected to the panel and surrounding the fenestration, wherein the second antenna is placed on the inner surface of the panel.

6. The surgical drape according to claim 5, wherein the second antenna is electrically connected to the first antenna.

7. The surgical drape according to claim 6, wherein the coils of the first antenna are laterally offset from coils of the second antenna.

8. The surgical drape according to claim 3, wherein the antenna is imbedded in the outer surface of the panel.

9. The surgical drape according to claim 8, wherein the antenna is a first antenna, and the surgical drape includes a second antenna connected to the panel and surrounding the fenestration, wherein the second antenna is imbedded in the inner surface of the panel.

10. The surgical drape according to claim 9, wherein the second antenna is electrically connected to the first antenna.

11. The surgical drape according to claim 10, wherein the coils of the first antenna are laterally offset from coils of the second antenna.

12. An interrogation and detection system for use on a patient in a surgical environment, comprising:
a controller;
a medical device having a transponder configured for storing information thereon; and
a surgical drape configured for in situ deployment on the patient while in the surgical environment, the surgical drape including:
a panel having an outer surface and an inner surface;
a fenestration centrally located in and extending through the panel; and
an antenna connected to the panel and connectable to the controller, the antenna surrounding the fenestration, wherein the antenna is configured to interrogate an area of the patient that is in proximity to the fenestration in response to control signals received from the controller, and wherein the antenna is configured to receive response signals from the transponder of the medical device and transmit the response signals to the controller for processing.

13. The interrogation and detection system according to claim 12, wherein the antenna is a coil of conductive material.

14. The interrogation and detection system according to claim 13, wherein the antenna is planar.

15. The interrogation and detection system according to claim 14, wherein the antenna is placed on the outer surface of the panel.

16. The interrogation and detection system according to claim 15, wherein the antenna is a first antenna, and the surgical drape includes a second antenna connected to the panel and surrounding the fenestration, wherein the second antenna is placed on the inner surface of the panel, and wherein the controller controls each of the first and second antennas.

17. The interrogation and detection system according to claim 16, wherein the second antenna is electrically connected to the first antenna, and wherein the coils of the first antenna are laterally offset from coils of the second antenna.

18. The interrogation and detection system according to claim 14, wherein the antenna is imbedded in the outer surface of the panel.

19. The interrogation and detection system according to claim 18, wherein the antenna is a first antenna, and the surgical drape includes a second antenna connected to the panel and surrounding the fenestration, wherein the second antenna is imbedded in the inner surface of the panel, and wherein the controller controls each of the first and second antennas.

20. The interrogation and detection system according to claim 19, wherein the second antenna is electrically connected to the first antenna, and wherein the coils of the first antenna are laterally offset from coils of the second antenna.
